(19) United States
(12) Patent Application Publication
(10) Pub. No.: US 2013/0116441 A1
(43) Pub. Date: May 9, 2013

(54) INTERMEDIATES AND PROCESS FOR PREPARING A THROMBIN SPECIFIC INHIBITOR

(71) Applicants: Antoni Segade Rodríguez, Barcelona (ES); Mireia Pastó Aguilà, Barcelona (ES)

(72) Inventors: Antoni Segade Rodríguez, Barcelona (ES); Mireia Pastó Aguilà, Barcelona (ES)

(73) Assignee: ESTEVE QUIMICA, S.A., BARCELONA (ES)

(21) Appl. No.: 13/809,391

(22) PCT Filed: Jul. 8, 2011
(86) PCT No.: PCT/EP11/61680

§ 371 Date: Jan. 9, 2013

(30) Foreign Application Priority Data
Jul. 9, 2010 (ES) P 201031048

(51) Int. Cl. C07D 401/12; C07D 213/75

(52) U.S. Cl. CPC C07D 401/12; C07D 213/75
USPC 546/273.4; 546/335

(57) ABSTRACT

Process for the preparation of a compound of formula (I), or a pharmaceutically acceptable salt thereof, wherein R¹ and R² represent H; or either R¹ represents ethyl and R² represents n-hexyloxycarbonyl that applies to industrial scale, novel intermediates useful for the preparation thereof, and processes of preparing said intermediates.

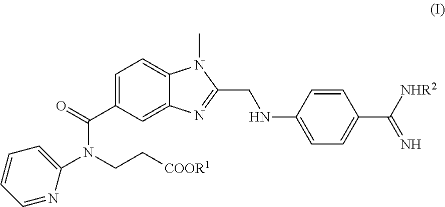

The present invention is related to a process for preparing dabigatran, dabigatran etexilate, as well as pharmaceutically acceptable salts thereof. It is also related to novel intermediates useful for the preparation thereof and processes of preparing said intermediates.

### BACKGROUND ART

Dabigatran is the generic name of compound N-[([(amidinophenyl)amino]methyl)-1-methyl-1H-benzimidazole-5-carbonyl]-N-(2-pyridyl)-3-aminopropionic acid, the chemical structure of which is the following:

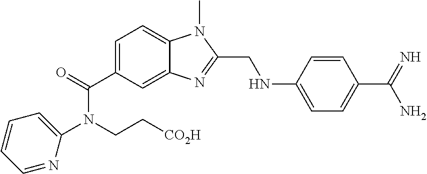 [SMILES: Cn1c(CNc2ccc(C(=N)N)cc2)nc2cc(C(=O)N(CCC(=O)O)c3ccccn3)ccc21]

Dabigatran is a thrombin specific inhibitor that is given orally in the form of prodrug dabigatran etexilate. The latest is rapidly absorbed after oral administration and converts to dabigatran, the pharmacologically active molecule, through hydrolysis catalyzed by plasma and liver esterases. The chemical name for dabigatran etexilate is ethyl N—[([([(N′-hexyloxycarbonyl)amidino]phenyl)amino]methyl)-1-methyl-1H-benzimidazole-5-carbonyl]-N-(2-pyridyl)-3-aminopropionate, and its chemical structure, the following:

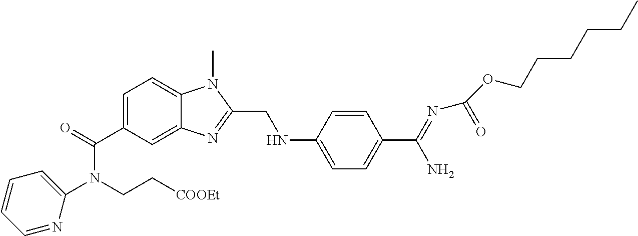 [SMILES: CCCCCCOC(=O)/N=C(\N)c1ccc(NCc2nc3cc(C(=O)N(CCC(=O)OCC)c4ccccn4)ccc3n2C)cc1]

Dabigatran and dabigatran etexilate were first described in patent application WO 98/37075. Several dabigatran etexilate salts, including the mesylate, have been described in documents WO 03/74056, WO 2006/114415 and WO 2008/43759.

Two synthesis pathways have mainly been described for preparing dabigatran and dabigatran etexilate. The first process described in application WO 98/37075 is based on the following synthesis scheme:

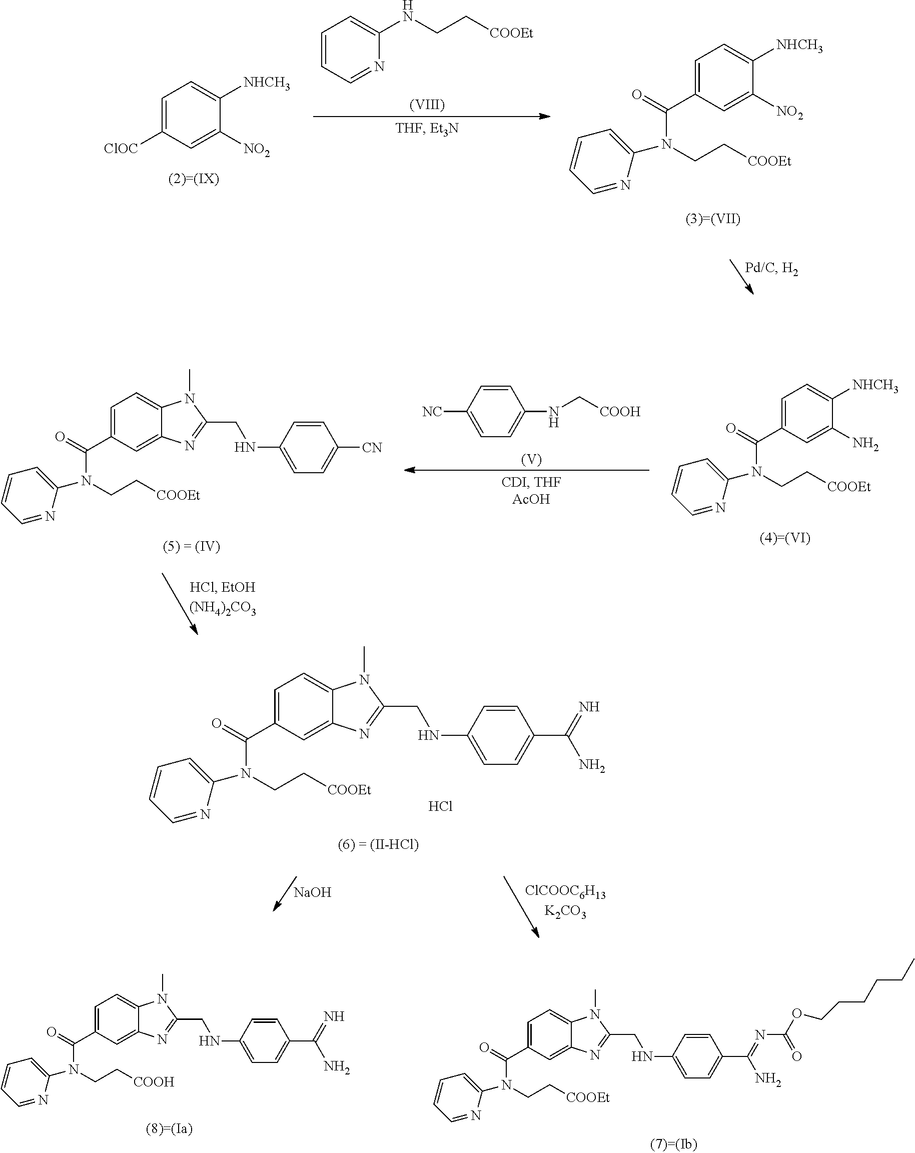

The second process has been described, e.g. in document WO 2006/000353 and is based on the following synthesis scheme:

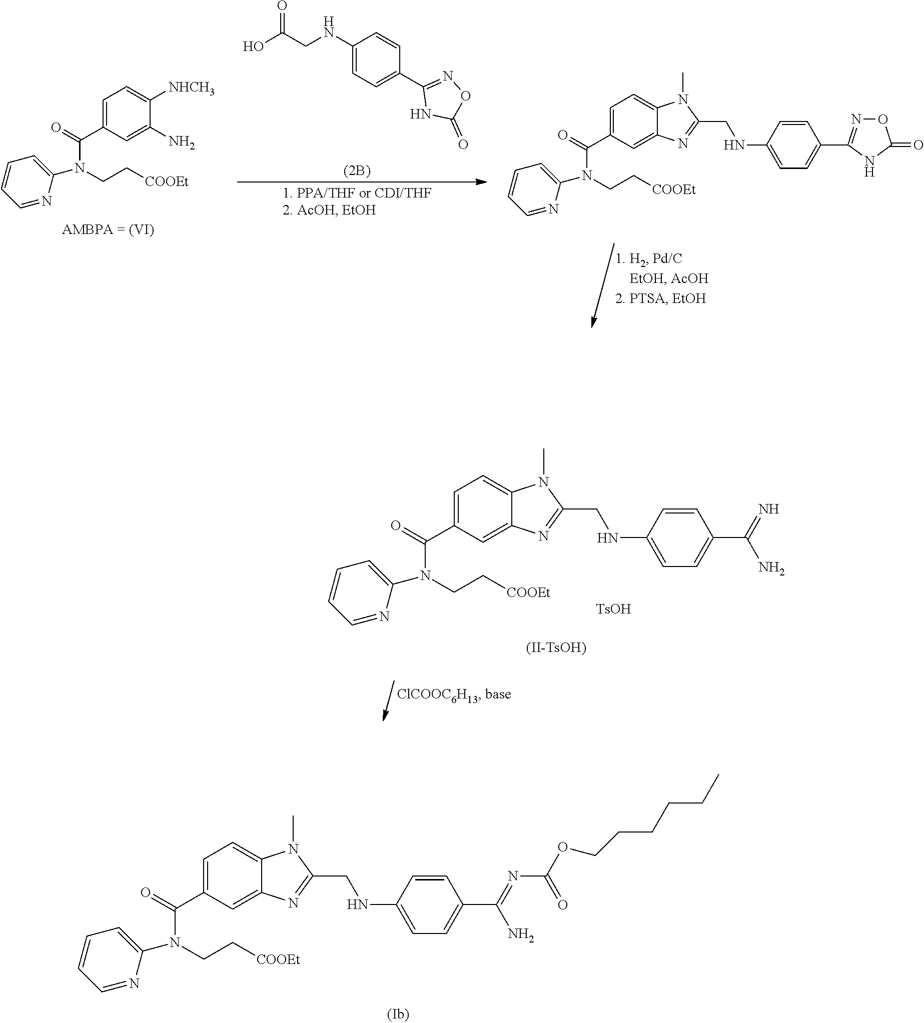

Both synthesis schemes have a common diamino intermediate, ethyl N-(3-amino-4-methylaminobenzoyl)-N-(2-pyridyl)-3-aminopropionate (VI), designated as compound (4) in document WO 98/37075 and as AMBPA in document WO 2006/000353, formed from the corresponding nitrocompound (VII). Compound (VI) results to be a key intermediate in the preparation of dabigatran and dabigatran etexilate.

The preparation of nitrocompound (VII) according to the previous documents shows some drawbacks. In particular, the preparation of this intermediate requires chromatographic purification, thereby this step is not convenient to be carried out at an industrial level.

Further, the catalytic hydrogenation of nitrocompound (VII) to obtain the diamino intermediate (VI) described in WO 98/37075 shows problems in scaling up, giving dirty and incomplete reactions, as the applicant himself indicates in document WO 2009/153214. This patent application describes catalytic hydrogenation of compound of formula (VII) in the presence of a tertiary amine, preferably trimethylamine, triethylamine, diisopropylethylamine and 1,8-diazabicyclo[5.4.0]undec-7-ene (DBU).

On the other side, in document WO 2009/111997 compound (VII) hydrochloride is described.

Therefore, a need exists of having alternative processes for the preparation of dabigatran and dabigatran etexilate, in particular if they are easy to industrialize.

### DESCRIPTION OF THE INVENTION

The inventors have found a new process for preparing dabigatran and dabigatran etexilate easy to industrialize, that courses with high yield and purity and overcomes the drawbacks described above.

On the other side, the inventors have also found novel solid forms of a key intermediate that show high purities and contribute to the optimization of the process for preparing dabigatran. The isolation of this intermediate in these solid forms is advantageous in that allows to obtain a final product with higher purity without the need of chromatographic purification.

Thus, a first aspect of the invention relates to a process for preparing a compound of formula (I), or a pharmaceutically acceptable salt thereof, or a pharmaceutically acceptable solvate thereof, including a hydrate,

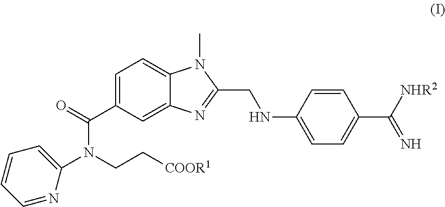

wherein R¹ and R² represent H; or either R¹ represents ethyl and R² represents n-hexyloxycarbonyl, comprising

a) catalytically hydrogenating the compound of formula (VII)

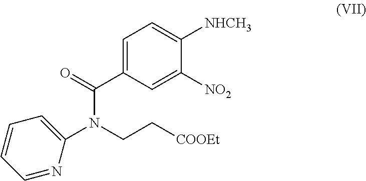 [SMILES: CCOC(=O)CCN(C(=O)c1ccc(NC)c([N+](=O)[O-])c1)c1ccccn1]

in the presence of an inorganic base and within a solvent, to obtain the compound of formula (VI); and

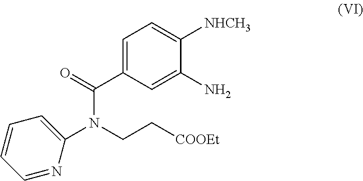 [SMILES: CCOC(=O)CCN(C(=O)c1ccc(NC)c(N)c1)c1ccccn1]

b) converting the compound of formula (VI) obtained into a compound of formula (I) or a pharmaceutically acceptable salt thereof, or a pharmaceutically acceptable solvate thereof, including a hydrate.

The presence of an inorganic base in the catalytic hydrogenation step overcomes the drawbacks of the state of the art mentioned above. Thus, on one side, and referring to the process described in document WO 98/37075, the use of an inorganic base allows a complete conversion in a reasonable time and yields cleaner reaction crudes.

Additionally, the use of an inorganic base also shows advantages over the tertiary amines described in the patent application WO 2009/153214 or over the use of a secondary amine, as e.g. diisopropylamine, or pyridine. Thus, the inorganic bases are of general use, less toxic and less expensive than amines, and also easier to remove by filtration.

In a preferred embodiment, the inorganic base is selected from hydroxides, carbonates and phosphates of alkaline and alkaline earth metals, preferably carbonates or phosphates. In another preferred embodiment, the inorganic base is selected from NaOH, KOH, Na₂CO₃, K₂CO₃, (NH₄)₂CO₃, NaHCO₃, KHCO₃, Na₃PO₄, NaH₂PO₄, Na₂HPO₄, K₃PO₄, KH₂PO₄, and K₂HPO₄. In a more preferred embodiment, the inorganic base is K₂CO₃ or K₃PO₄.

Generally, the inorganic base amount is 0.05-10% by weight to the starting nitrocompound of formula (VII), preferably between 2-8%, and more preferably 5%.

The catalytic hydrogenation reaction is carried out in the presence of a catalyst and within a suitable solvent. As solvent can be used protic solvents, including (C₁-C₆)alcohols; aprotic solvents, as e.g. (C₃-C₆)ethers, (C₁-C₆)alkyl (C₁-C₆)esters, (C₃-C₆)amides; and/or mixtures thereof with or without water. Examples of solvents include, without being limited to methanol, ethanol, n-propanol and isopropanol, tetrahydrofuran, dimethoxyethyl ether, dimethylformamide, N-methylpyrrolidone, toluene or ethyl acetate. Preferably, the solvent used is ethyl acetate.

In general, the hydrogenation is brought to a temperature of between 10-100° C., preferably between 20-80° C., more preferably between 50-60° C.; and under a pressure of between 0.5-10 bar, preferably between 2-6 bar, and more preferably at about 4 bar.

The hydrogenation catalyst is, in general, a transition metal as nickel, platinum or palladium, or a salt or oxide thereof, preferably Raney nickel, platinum oxide and palladium over an inert material, as e.g. carbon. Preferably, the catalyst is Pd/C. In a preferred embodiment, the amount of Pd/C is 2-20%, more preferably 5%.

In a preferred embodiment, previous to the catalytic hydrogenation step:

(i) the compound of formula (IX) is reacted,

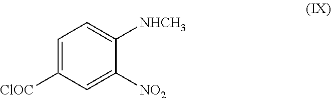

with the compound of formula (VIII)

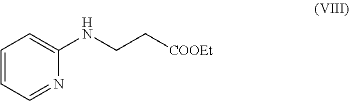 [SMILES: CCOC(=O)CCNc1ccccn1]

in the presence of a base;

(ii) the product obtained is reacted with hydrobromic acid to yield the compound of formula (VII-HBr); and

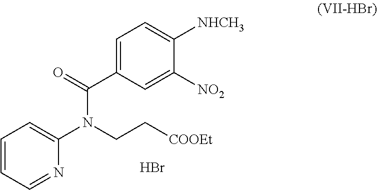 [SMILES: CCOC(=O)CCN(C(=O)c1ccc(NC)c([N+](=O)[O-])c1)c1ccccn1 ; Br]

(iii) the compound of formula (VII-HBr) is reacted with a base to yield the compound of formula (VII).

The starting compound of formula (IX) may be found in the form of a free base or a salt thereof.

The coupling reaction between the compound of formula (IX) and the compound of formula (VIII) is already known in the state of the art, e.g. in the patent application WO 98/37075. This reaction can be carried out within a suitable solvent, as e.g. tetrahydrofuran, at a suitable temperature, preferably room temperature, and preferably in the presence of a base, such as triethylamine.

However, unlike WO 98/37075, in the present invention, the obtained compound of formula (VII) is not purified by chromatography, but after the work-up it precipitates in the form of the corresponding hydrobromide (VII-HBr).

The standard precipitation process takes place within a solution of the compound of formula (VII) in a suitable solvent, at a temperature between 10-60° C., preferably at room temperature, by adding HBr in pure gas form, or in aqueous solution or in an organic solution, preferably HBr in aqueous solution or in an organic solution, and more preferably 48% aqueous HBr.

Generally, the solvent of the HBr organic solutions can be a (C₁-C₆)alcohol, such as ethanol, isopropanol or butanol; a (C₁-C₆)alkyl (C₁-C₆)ester, such as ethyl acetate, isopropyl acetate or isobutyl acetate; a (C₃-C₈)ketone, such as methylisobutylketone, methylethylketone or acetone; a (C₃-C₆)ether such as methyl tert-butyl ether, 2-methyltetrahydrofuran, or tetrahydrofuran; a (C₁-C₆)halogenated solvent, such as dichloromethane; a (C₅-C₁₂)alkane such as heptane, (C₅-C₁₂)cycloalkane such as cyclohexane; a (C₁-C₆)carboxylic acid such as acetic acid, or mixtures of the above.

There are typically used between 0.8-1.5 equivalents of HBr in relation to the starting compound (VII), preferably between 1.1-1.2 equivalents.

The solvent in which the compound of formula (VII) is dissolved can be a (C₁-C₆)alcohol, such as ethanol, isopropanol or butanol; a (C₁-C₆)alkyl (Cᵣ, C₆)ester, such as ethyl acetate, isopropyl acetate or isobutyl acetate; a (C₃-C₈)ketone, such as methylisobutylketone, methylethylketone or acetone; a (C₃-C₆)ether such as methyl tert-butyl ether, 2-methyltetrahydrofuran, or tetrahydrofuran; a (C₁-C₆)halogenated solvent, such as dichloromethane; a (C₆-C₉)aromatic solvent such as toluene or xylene; a (C₅-C₁₂)alkane such as heptane, (C₅-C₁₂)cycloalkane such as cyclohexane, or mixtures of the above. Preferably, the solvent is (C₃-C₆)ether, and more preferably, tetrahydrofuran.

After the appearance of the solid corresponding to the hydrobromide (VII-HBr), the reaction mixture is stirred for some time, generally between 0-3 hours, preferably 30 minutes, keeping the temperature indicated above. Subsequently, the mixture can optionally be stirred at 0° C. for some time, generally between 0-3 hours, preferably 30 minutes, and filtered. In a preferred embodiment, the reaction mixture is stirred between 30 minutes and 3 hours at room temperature and, subsequently, between 30 minutes and 3 hours at 0° C. Finally, the solid is filtered out, washed and dried, obtaining compound (VII-HBr). The solid obtained can optionally be recrystallized from ethanol, obtaining the product with a higher than 99% a/a purity according to HPLC/MS.

The compound of formula (VII-HBr) may be converted into the compound of formula (VII) by reactions well known to the skilled in the art. For example, the compound of formula (VII-HBr) is reacted with an organic base such as triethylamine, diethylamine or diisopropylethylamine, or with an inorganic base such as NaOH, KOH, Na₂CO₃, K₂CO₃, NaHCO₃, KHCO₃, Na₃PO₄, or K₃PO₄. Generally, between 1-3 equivalents of base are used in relation to the starting hydrobromide, preferably 1.15 equivalents.

This reaction takes place within an organic solvent optionally mixed with water. Examples of solvents are (C₃-C₆)ethers, such as dioxane or tetrahydrofuran; (C₃-C₈)ketones such as methylisobutylketone or methylethylketone, (C₁-C₆)halogenated solvents as dichloromethane; or (C₁-C₆)alkyl (C₁-C₆)esters as ethyl acetate. Preferably, the reaction takes place in dichloromethane and aqueous NaOH.

Another aspect of the invention relates to the compound of formula (VII-HBr), i.e. ethyl N-(4-methylamino-3-nitrobenzoyl)-N-(2-pyridyl)-3-aminopropionate hydrobromide. In a preferred embodiment, the invention relates to the compound of formula (VII-HBr) in solid form, including any crystalline or amorphous form. In a more preferred embodiment, the invention relates to the compound of formula (VII-HBr) in crystalline form.

In another preferred embodiment, the invention relates to the crystalline form I of compound of formula (VII-HBr) that shows an X-Ray powder diffraction pattern substantially according to FIG. 1. In another preferred embodiment, the invention relates to the crystalline form I of compound of formula (VII-HBr) that shows a X-Ray powder diffraction pattern comprising 2θ angle values at 8.0, 11.8, 12.1, 12.8, 14.6, 16.1, 17.6, 18.3, 20.3, 21.4, 23.8, 24.7, 25.0 and 27.3, measured in a X-ray diffractometer with Cu Kα radiation (1.5418 Å).

It is also part of the invention the process for preparing the crystalline form I. This crystalline form may be obtained by a process comprising reacting the compound (VII) with HBr in tetrahydrofuran and water, and separating the crystallized product from the reaction medium, e.g. by filtration. Alternatively, this crystalline form may be obtained by recrystallizing compound (VII-HBr) from a solution thereof in tetrahydrofuran and water, at a temperature comprised between 10-60° C., and in a concentration between 3-15 volumes of solvent, preferably between 4-7 volumes of solvent. Generally, the water percentage in the tetrahydrofuran may be between 1-10%, preferably between 4-8%; and the crystallized product is filtered out at a temperature that may range between −20° C. and room temperature.

Optionally, in any of the two foregoing processes the solution of compound (VII-HBr) may be seeded to facilitate the beginning of crystallization. In this particular embodiment, the solution is seeded with compound (VII-HBr) form I previously obtained by the process without seeding.

In another preferred embodiment, the invention relates to the crystalline form II of compound of formula (VII-HBr) that shows an X-Ray powder diffraction pattern substantially according to FIG. 2. In another preferred embodiment, the invention relates to the crystalline form II of compound of formula (VII-HBr) that shows a X-Ray powder diffraction pattern comprising 2θ angle values at 9.2, 11.8, 18.0, 19.3, 20.2, 23.5, 24.7, 26.0, 28.4, 28.8, 29.6 and 30.4, measured in a X-ray diffractometer with Cu Kα radiation (1.5418 Å).

It is also part of the invention the process for preparing the crystalline form II. This crystalline form may be obtained by a process comprising crystallization of compound (VII-HBr) from a solution thereof in ethanol, at a temperature comprised between 10-80° C., and in a concentration between 2-15 volumes of ethanol, preferably between 4-7 volumes of solvent. Generally, the crystallized product is filtered out at a temperature that may range between −5° C. and room temperature.

The compound (VII-HBr) in solid form has the advantage that is particularly easy to separate by filtration. This characteristic has a direct effect on the global yield of the process and, therefore, is specially important when the process is carried out at an industrial scale, since a product showing improved separation characteristics can be isolated faster, better washed and therefore faster dried, and obtained in a higher degree of purity.

The formation of the hydrobromide by the addition of an aqueous solution of HBr onto a solution of the free base allows obtaining the compound (VII-HBr) with a higher yield and higher purity, further allowing a good separation of the mother liquors by filtration.

Further, the compound (VII-HBr) is also advantageous in relation to the hydrochloride already described in application WO 2009/111997, since when it is obtained using an aqueous acid solution (aqueous concentrated HCl (37%)), which is more convenient from the industrial point of view, the product obtained tends to retain part of the mother liquors, hindering its filtration and drying.

The preparation of compound of formula (I) or a salt thereof from the compound of formula (VI) it is already known in the state of the art. Mainly, two synthesis strategies may be followed. The first of them comprises the coupling of the compound of formula (VI) with the compound of formula (V) that comprises a cyano group;

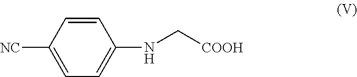 [SMILES: N#Cc1ccc(NCC(=O)O)cc1]

to obtain the compound of formula (IV)

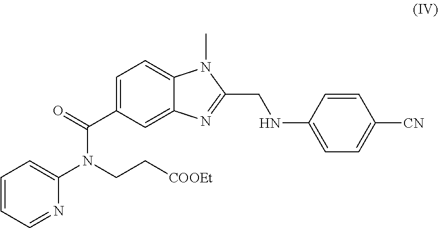 [SMILES: CCOC(=O)CCN(C(=O)c1ccc2c(c1)nc(CNc1ccc(C#N)cc1)n2C)c1ccccn1]

which is optionally converted into a salt thereof by reaction with the corresponding acid.

The Pinner reaction of the compound of formula (IV) or a salt thereof, i.e., the conversion of the cyano group to imidate and later conversion to amidine results in the compound of formula (II)

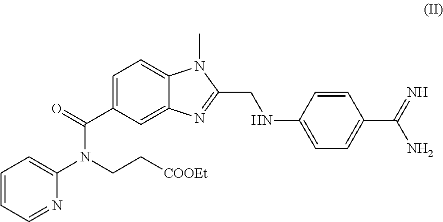 [SMILES: CCOC(=O)CCN(C(=O)c1ccc2c(c1)nc(CNc1ccc(C(=N)N)cc1)n2C)c1ccccn1]

which is optionally converted into a salt thereof by reaction with the corresponding acid, and is subsequently converted in a compound of formula (I).

The conversion of the compound of formula (IV) in the compound of formula (II) is carried out in the presence of hydrochloric acid in ethanol, and subsequent addition of ammonia or an ammonium salt.

Alternatively, the compound of formula (VI) can be reacted with a compound comprising an oxadiazolone group, such as e.g. the compound of formula (X)

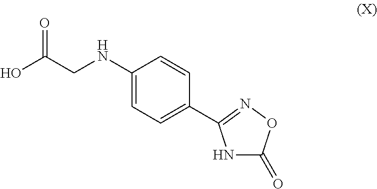 [SMILES: O=C(O)CNc1ccc(-c2noc(=O)[nH]2)cc1]

to obtain the compound of formula (XI)

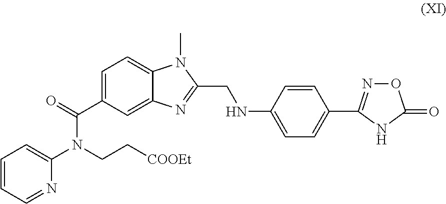 [SMILES: CCOC(=O)CCN(C(=O)c1ccc2c(c1)nc(CNc1ccc(-c3noc(=O)[nH]3)cc1)n2C)c1ccccn1]

The catalytic hydrogenation of the compound of formula (XI) results in the compound of formula (II), which is converted into a compound of formula (I).

The catalytic hydrogenation is carried out, e.g. using Pd/C as catalyst, in a solvent such as ethanol in the presence of acetic acid.

The formation of the benzimidazole ring by reaction between the compound of formula (VI) and the compound of formula (V), or between the compound of formula (VI) and the compound of formula (X), to obtain the compound of formula (IV) or the compound of formula (XI), respectively, can be carried out e.g. in the presence of a coupling agent such as 1,1′-carbonyldiimidazole or the anhydride of propanephosphonic acid in tetrahydrofuran; and subsequent cyclization with a cyclization agent, as e.g. acetic acid in ethanol.

In a more preferred embodiment, the compound of formula (VI) is converted into the compound of formula (IV) by reaction with the compound of formula (V) and, subsequently, the compound of formula (IV) is converted into the compound of formula (II).

Each of the process steps in the present invention represents a significant improvement in relation to the processes described and may be combined with some of the steps already known. Additionally, when the different steps in the present invention are carried out together the resulting process is a particularly effective industrializable process.

As already mentioned above, the preparation of the compounds of formula (I) from the compound of formula (II) is already known in the state of the art, e.g. in the patent application WO 98/37075.

By the process of the invention a compound of formula (I) may be obtained, wherein R¹ represents H and R² represents H, i.e. a compound (Ia),

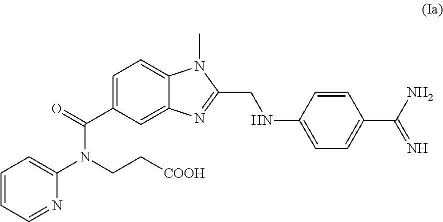 [SMILES: Cn1c(CNc2ccc(C(=N)N)cc2)nc2cc(C(=O)N(CCC(=O)O)c3ccccn3)ccc21]

corresponding to dabigatran, or either a compound of formula (I), wherein R¹ represents ethyl and R² represents n-hexyloxycarbonyl, wherein the n-hexyloxycarbonyl radical refers to the radical —COO—(CH₂)₅CH₃, i.e. a compound (Ib),

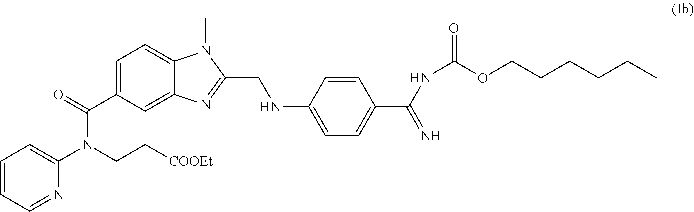 [SMILES: CCCCCCOC(=O)NC(=N)c1ccc(NCc2nc3cc(C(=O)N(CCC(=O)OCC)c4ccccn4)ccc3n2C)cc1]

corresponding to dabigatran etexilate.

By way of example, the compound of formula (Ib) may be prepared by reaction of the compound of formula (II) with an n-hexyl haloformate of formula (XI)

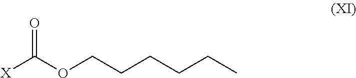

wherein X is a halogen such as Cl or Br, preferably Cl. The reaction is carried out at a temperature between 0-50° C., preferably between 10-25° C. and in the presence of a base, such as K₂CO₃, Na₂CO₃, KHCO₃, NaHCO₃, or triethylamine. In a preferred embodiment K₂CO₃ is used. In another preferred embodiment, triethylamine is used. This reaction can be carried out in a (C₃-C₈)ketone-type solvent, such as acetone or (C₃-C₆)ether type, such as dioxane or tetrahydrofuran, optionally in the presence of water. Preferably, this reaction is carried out in tetrahydrofuran or acetone.

The compound of formula (Ia) may be prepared by a hydrolysis reaction of the compound of formula (II). Generally, the hydrolysis is carried out in the presence of a base, such as sodium hydroxide, in a suitable solvent, as e.g. a mixture of ethanol and water, and at a suitable temperature, e.g. room temperature.

On the other side, a compound of formula (I) may be converted into a pharmaceutically acceptable salt thereof by treatment with an acid, or either a pharmaceutically acceptable salt of the compound of formula (I) may be converted into a compound of formula (I) by treatment with a base, or either a salt of the compound of formula (I) may be converted into another salt of the compound of formula (I) by ion exchange.

The salts of the compound of formula (I), in particular of the compound (Ib), and the obtaining thereof have already been described, e.g. in the documents WO 03/074056, WO 2006/114415 and WO 2008/43759. Likewise, a salt of the compound of formula (I) may be converted into another salt of the compound of formula (I) by ion exchange.

In a preferred embodiment, the invention relates to the compound (Ib) methanesulfonate or mesylate, i.e. to dabigatran etexilate mesylate (Ib-MsOH). This salt is prepared from the compound (Ib) and methanesulfonic acid, e.g. in a mixture of acetone and water, and at a temperature between 20-40° C.

The solvates of the compounds of formula (I) or of its pharmaceutically acceptable salts, including hydrates, are also part of the invention. In general, the solvated forms with pharmaceutically acceptable solvents such as water, ethanol and the like are equivalent to the non-solvated forms for the purposes of the present invention. Methods of solvation, for instance, crystallization in the presence of the solvent of solvation, are generally known in the art.

Throughout the description and claims the word “comprise” and variations of the word, are not intended to exclude other technical features, additives, components, or steps. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### BRIEF DESCRIPTION OF THE DRAWINGS

FIG. 1 shows the X-Ray powder diffraction curve (intensity (counts) vs. 2theta angle (°)) of the crystalline form I of the compound of formula (VII-HBr).

FIG. 2 shows the X-Ray powder diffraction curve (intensity (counts) vs. 2theta angle (°)) of the crystalline form II of the compound of formula (VII-HBr).

### EXAMPLES

In the examples, the following abbreviations have been used:

EtOAc: ethyl acetate

Ar: argon

c.: concentrated

t.l.c.: thin layer chromatography

DMF: dimethylformamide

EtOH: ethanol

Et₃N: triethylamine

PXRD: Powder X-Ray diffraction

r.t.: room temperature

THF: tetrahydrofuran

DRX analysis was performed in a PANalytical X'Pert PRO MPD diffractometer with Bragg-Bentano geometry and Cu K_{α} radiation (1.5418 Å). The system was provided with a RTMS detector. Samples were grinded and placed in Si sample holders of zero background. The recording parameters were a range of 2Theta=3-40° and a total recording time of 125 s.

### Example 1

### Ethyl N-(4-methylamine-3-nitrobenzoyl)-N-(2-pyridyl)-3-aminopropionate hydrobromide (VII-HBr)

4-(Methylamine)-3-nitrobenzoic acid hydrochloride (13.52 g, 58.1 mmol) was suspended in SOCl₂ (105 mL, 1.44 mol), anhydrous N,N-dimethylformamide (DMF) (0.55 mL) was added and refluxed for 45 minutes. After leaving the orange solution to cool down, the SOCl₂ was distilled at low pressure. Next, the yellow solid residue obtained was suspended in toluene (40 mL) and the solvent was distilled at low pressure. This operation was performed three times.

The solid obtained was suspended under Ar in anhydrous THF (80 mL) and Et₃N (20.2 mL, 144.9 mmol) was slowly added. Next, it was chilled down to 0° C., an ethyl N-(2-pyridyl)-3-aminopropionate (VIII) solution (11.3 g, 58.1 mmol) was slowly added in anhydrous THF (42 mL) and stirred at r.t. overnight. The solvent was distilled at low pressure, the residue was redissolved in CH₂Cl₂ (140 mL) and washed with H₂O (85 mL). The orange aqueous phase was extracted with CH₂Cl₂ (2×28 mL). The organic phases were mixed, washed with NaOH 1 N (24 mL) and dried over anhydrous MgSO₄, and the solvent was distilled at low pressure.

The brown oil obtained was dissolved in THF (120 mL) and 48% HBr (7.60 mL, 67.2 mmol) was added dropwise. After a short time an abundant yellow solid appeared. The suspension was stirred at r.t for 30 min, and after at 0° C. for 1 h. The solid was filtered out, washed with THF (10 mL) and dried under vacuum, obtaining the crude compound (VII-HBr) corresponding to crystalline form I (22.16 g, 84% yield, 91.2% a/a purity according to HPLC/MS).

¹H RMN (400 MHz, d₆-DMSO): δ (ppm)=8.42 (ddd, J=4.8, 1.6, 0.8, 1H), 8.34 (bs, 1H), 7.91 (d, J=2.4, 1H), 7.69 (ddd, J=8.0, 7.6, 1.6, 1H), 7.31 (dd, J=8.8, 2.0, 1H), 7.20 (ddd, J=7.6, 4.8, 0.8, 1H), 7.07 (d, J=8.0, 1H), 6.82 (d, J=8.8, 1H), 4.16 (t, J=7.2, 2H), 3.95 (q, J=7.2, 2H), 2.89 (s, 3H), 2.64 (t, J=6.8, 2H), 1.10 (t, J=7.2, 3H).

Melting point (Tₘₑₗₜ): 165-166° C.

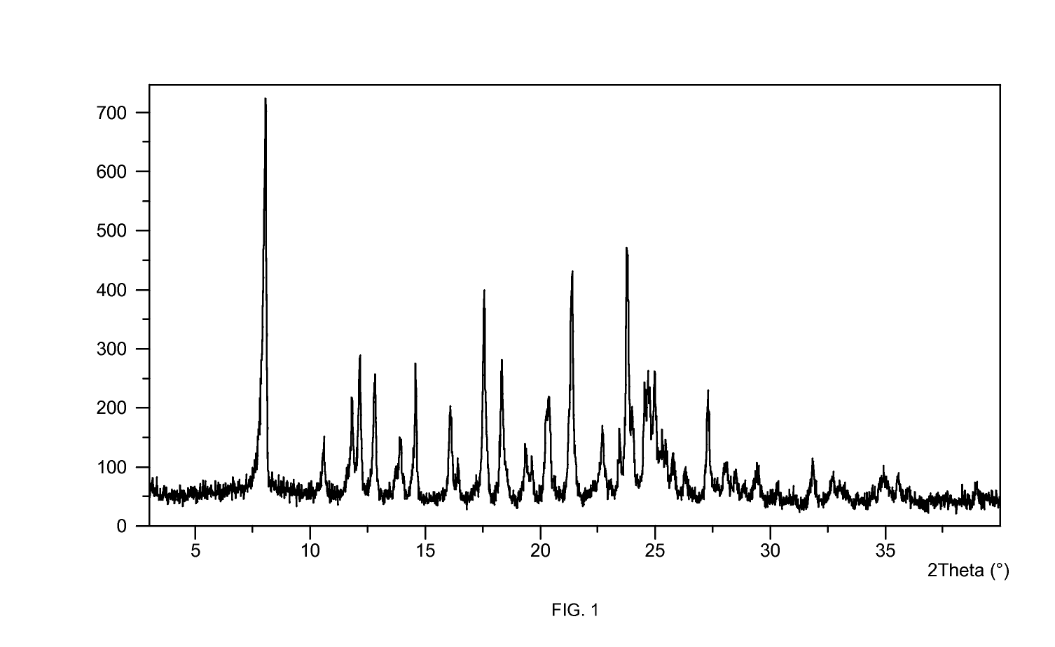

PXRD: FIG. 1, 2theta angle values (°): 8.0, 11.8, 12.1, 12.8, 14.6, 16.1, 17.6, 18.3, 20.3, 21.4, 23.8, 24.7, 25.0 and 27.3.

The crystalline form I was recrystallized from EtOH (95 mL), filtered out, washed with EtOH (10 mL) and dried under vacuum, obtaining crystalline form II of compound (VII-HBr) (18.61 g, 71% global yield, 100% a/a purity according to HPLC/MS).

Melting point (Tₘₑₗₜ): 169-170° C.

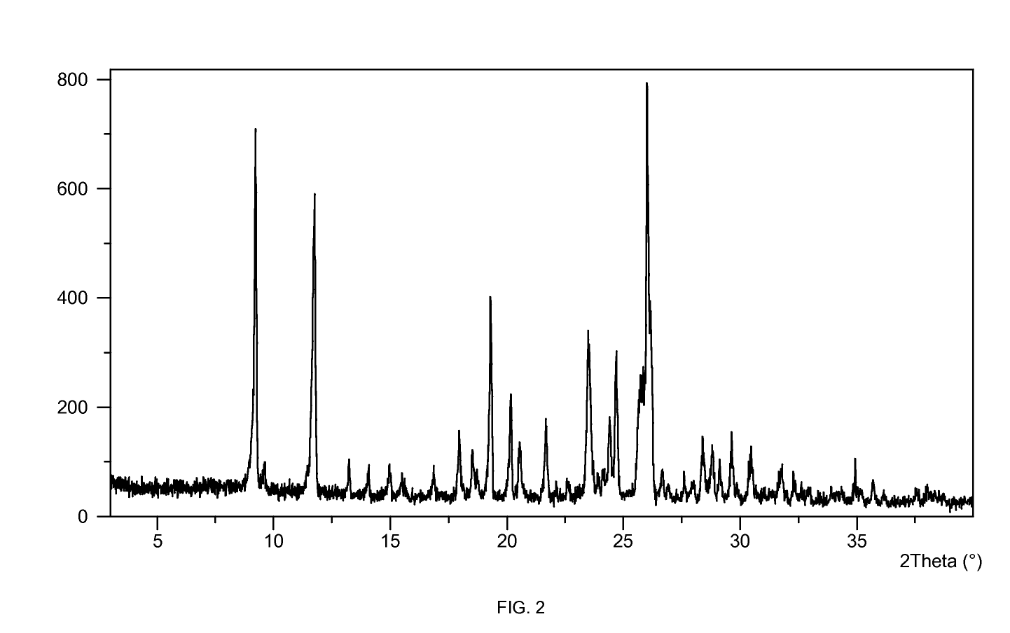

PXRD: FIG. 2, 2theta angle values (°): 9.2, 11.8, 18.0, 19.3, 20.2, 23.5, 24.7, 26.0, 28.4, 28.8, 29.6 and 30.4.

### Example 2

### Ethyl N-(3-amino-4-methylaminobenzoyl)-N-(2-pyridyl)-3-aminopropionate (VI)

### a) Reaction in the Presence of K₂CO₃

The hydrobromide (VII-HBr) (12.00 g, 26.5 mmol) was suspended in CH₂Cl₂ (60 mL) and NaOH 1N (30 mL) and was stirred until complete dissolution of the solid was observed. The organic phase was separated and the aqueous phase extracted with CH₂Cl₂ (10 mL). The two organic phases were mixed, dried over anhydrous MgSO₄, the solvent was distilled at low pressure and dried under vacuum.

The residue obtained (9.42 g) was dissolved in EtOAc (56 mL) and hydrogenated in the presence of K₂CO₃ (0.49 g, 5% by weight) and 5% Pd/C (0.96 g, 51.1% humidity, 5% by weight) in a 250 mL miniclave reactor at an initial pressure of 4 bar and 55° C. H₂ was refilled until complete conversion was observed by t.l.c. (cyclohexane:EtOAc 1:1). It was then left to cool down, it was filtered, the solid was washed with EtOAc (2×10 mL), the filtrate solvent was distilled at low pressure and dried under vacuum, obtaining compound (VI) (8.49 g, 94% yield, 99% a/a purity according to HPLC/MS).

¹H RMN (400 MHz, CDCl₃): δ (ppm)=8.43 (ddd, J=4.8, 1.6, 0.8, 1H), 7.39 (ddd, J=8.0, 7.2, 2.0, 1H), 7.00 (ddd, J=7.6, 5.2, 0.8, 1H), 6.85 (d, J=2.0, 1H), 6.76-6.70 (m, 2H), 6.33 (d, J=8.4, 1H), 4.37 (t, J=7.2, 2H), 4.06 (q, J=7.2, 2H), 3.12 (bs, 3H), 2.80 (s, 3H), 2.64 (t, J=7.2, 2H), 1.20 (t, J=7.2, 3H).

### b) Reaction in the Presence of K₃PO₄

The hydrobromide (VII-HBr) (2.00 g, 4.41 mmol) was suspended in CH₂Cl₂ (10 mL) and NaOH 1 N (5 mL) and was stirred until complete dissolution of the solid was observed. The organic phase was dried over anhydrous MgSO₄, the solvent was distilled at low pressure and dried under vacuum.

The residue obtained (1.46 g) was dissolved in EtOAc (8.5 mL) and hydrogenated in the presence of K₃PO₄ (0.07 g, 5% by weight) and 5% Pd/C (0.30 g, 51.1% humidity, 10% by weight) in a 250 mL miniclave reactor at an initial pressure of 4 bar and 55° C. After 1 h and 25 min it was left to cool down, it was filtered, the solid was washed with EtOAc (2×5 mL), the solvent was distilled at low pressure and dried under vacuum, obtaining compound (VI) (1.27 g, 84% yield, 100% a/a purity according to HPLC/MS).

### Example 3

### Ethyl N-(4-methylamino-3-nitrobenzoyl)-N-(2-pyridyl)-3-aminopropionate hydrobromide (VII-HBr) (obtaining form I with seeding)

4-(methylamino)-3-nitrobenzoic acid (20.0 g, 102 mmol) was suspended in SOCl₂ (156 mL, 2.14 mol), anhydrous DMF (0.80 mL) was added and refluxed for 45 minutes. After leaving the solution to cool down, the SOCl₂ was distilled at low pressure. Next, the yellow solid residue obtained was suspended in toluene (60 mL) and the solvent was distilled at low pressure. This operation was performed three times.

The solid obtained was suspended under Ar in anhydrous tetrahydrofuran (THF) (118 mL) and Et₃N (30 mL, 215 mmol) was slowly added. Next, it was chilled down to 0° C., an ethyl N-(2-pyridyl)-3-aminopropionate (VIII) solution (16.7 g, 86.0 mmol) in anhydrous THF (62 mL) was slowly added, and stirred at r.t. for 1 h and 30 minutes. The solvent was distilled at low pressure, the residue was redissolved in CH₂Cl₂ (207 mL) and washed with H₂O (46 mL) and NaOH 1 N (36 mL) and dried over anhydrous MgSO₄, and the solvent was distilled at low pressure.

The brown oil obtained (36.8 g) was dissolved in THF (167 mL). The precipitated triethylammonium chloride residues were filtered out, washed with THF (10 mL), and onto the mixed filtrates 48% HBr (11.2 mL, 98.9 mmol) was added dropwise for 5 minutes. Next it was seeded with VII-HBr (form I) and after a few moments the solid crystallized. The suspension was stirred at room temperature (r.t.) for 30 minutes and next in a water/ice bath for 1 h. The solid was filtered out, washed with THF (16 mL) and dried under vacuum at r.t., obtaining form I of VII-HBr (34.3 g, 88% yield from VIII, 93% a/a purity according to HPLC/MS).

1. A process for preparing a compound of formula (I), or a pharmaceutically acceptable salt thereof, 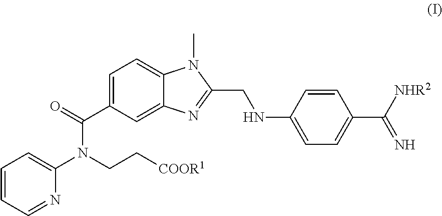
wherein R¹ and R² represent H; or either R¹ represents ethyl and R² represents n-hexyloxycarbonyl, comprising
a) catalytically hydrogenating the compound of formula (VII) 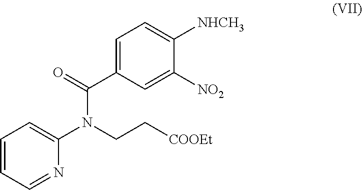 [SMILES: CCOC(=O)CCN(C(=O)c1ccc(NC)c([N+](=O)[O-])c1)c1ccccn1]
in the presence of an inorganic base and within a solvent, to obtain the compound of formula (VI); and 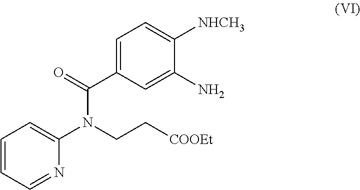 [SMILES: CCOC(=O)CCN(C(=O)c1ccc(NC)c(N)c1)c1ccccn1]
b) converting the compound of formula (VI) into a compound of formula (I) or a pharmaceutically acceptable salt thereof.

2. The process according to claim 1, wherein previously to step a):
(i) the compound of formula (IX) is reacted 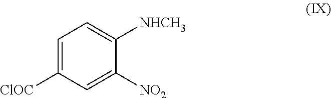
with the compound of formula (VIII) 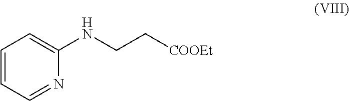 [SMILES: CCOC(=O)CCNc1ccccn1]
in the presence of a base;
(ii) the product obtained is reacted with hydrobromic acid to yield the compound of formula (VII-HBr); and 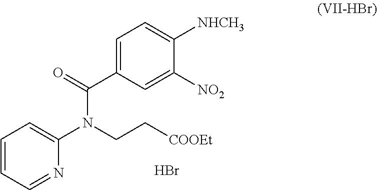 [SMILES: CCOC(=O)CCN(C(=O)c1ccc(NC)c([N+](=O)[O-])c1)c1ccccn1 ; Br]
(iii) the compound of formula (VII-HBr) is reacted with a base to yield the compound of formula (VII).

3. The process according to claim 2, wherein in step (ii) the hydrobromic acid is in 48% aqueous solution and the reaction is carried out in the presence of tetrahydrofuran.

4. The process according to claim 2, wherein the compound of formula (VII-HBr) is isolated as a solid.

5. The process according to claim 4, wherein the amount of inorganic base in step a) is 2-8% by weight in relation to the starting compound of formula (VII).

6. The process according to claim 4, wherein the inorganic base of step a) is K₂CO₃ or K₃PO₄.

7. The process according to claim 4, wherein the conversion of compound of formula (VI) into a compound of formula (I) or a pharmaceutically acceptable salt thereof is carried out by the following steps:
(b1) the compound of formula (VI) is reacted with the compound of formula (V) 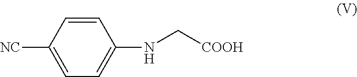 [SMILES: N#Cc1ccc(NCC(=O)O)cc1]
in the presence of a coupling agent and subsequent cyclization with a cyclization agent to yield the compound of formula (IV) 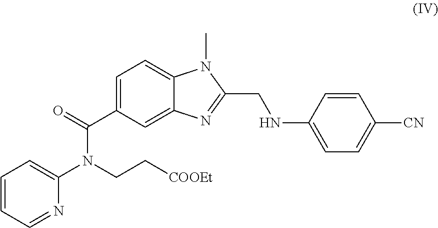 [SMILES: CCOC(=O)CCN(C(=O)c1ccc2c(c1)nc(CNc1ccc(C#N)cc1)n2C)c1ccccn1]
and, optionally, the compound of formula (IV) is converted into a salt thereof by reaction with the corresponding acid;
(b2) the product obtained in step (b1) is reacted in the presence of hydrochloric acid and ethanol, and subsequently ammonia or an ammonium salt is added to yield the compound of formula (II), 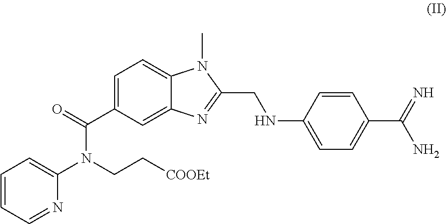 [SMILES: CCOC(=O)CCN(C(=O)c1ccc2c(c1)nc(CNc1ccc(C(=N)N)cc1)n2C)c1ccccn1]
and, optionally, the compound of formula (II) is converted into a salt thereof by reaction with the corresponding acid;
(b3) the compound of formula (II) or a salt thereof is converted into a compound of formula (I) by a hydrolysis reaction or by reaction with a n-hexyl haloformate in the presence of a base; and
(b4) optionally the compound of formula (I) is converted into a pharmaceutically acceptable salt thereof by treatment with an acid, or either a pharmaceutically acceptable salt of the compound of formula (I) is converted into a compound of formula (I) by treatment with a base, or either a salt of the compound of formula (I) is converted into another salt of the compound of formula (I) by ion exchange.

8. The process for preparing the compound of formula (VI) 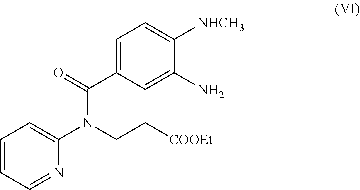 [SMILES: CCOC(=O)CCN(C(=O)c1ccc(NC)c(N)c1)c1ccccn1]
comprising catalytically hydrogenating the compound of formula (VII) 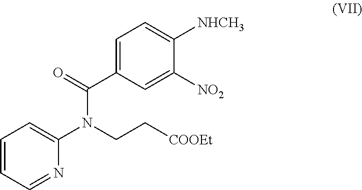 [SMILES: CCOC(=O)CCN(C(=O)c1ccc(NC)c([N+](=O)[O-])c1)c1ccccn1]
in the presence of an inorganic base and within a solvent.

9. The process according to claim 8, wherein previously:
(i) the compound of formula (IX) is reacted 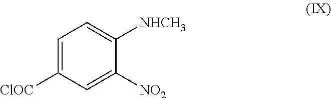
with the compound of formula (VIII) 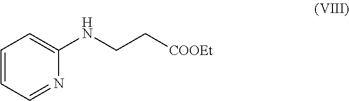 [SMILES: CCOC(=O)CCNc1ccccn1]
in the presence of a base;
(ii) the product obtained is reacted with hydrobromic acid to yield the compound of formula (VII-HBr); and 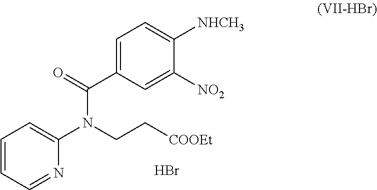 [SMILES: CCOC(=O)CCN(C(=O)c1ccc(NC)c([N+](=O)[O-])c1)c1ccccn1 ; Br]
(iii) the compound of formula (VII-HBr) is reacted with a base to yield the compound of formula (VII).

10. A compound of formula (VII-HBr), which is ethyl N-(4-methylamino-3-nitrobenzoyl)-N-(2-pyridyl)-3-aminopropionate hydrobromide.

11. The compound of formula (VII-HBr) according to claim 10, which is in crystalline form.

12. The compound of formula (VII-HBr) according to claim 11, which is in crystalline form I and shows a X-Ray powder diffraction pattern comprising 2θ angle values at 8.0, 11.8, 12.1, 12.8, 14.6, 16.1, 17.6, 18.3, 20.3, 21.4, 23.8, 24.7, 25.0 and 27.3, measured in a X-ray diffractometer with Cu Kα radiation (1.5418 Å).

13. The compound of formula (VII-HBr) according to claim 11, which is in crystalline form II and shows a X-Ray powder diffraction pattern comprising 2θ angle values at 9.2, 11.8, 18.0, 19.3, 20.2, 23.5, 24.7, 26.0, 28.4, 28.8, 29.6 and 30.4, measured in a X-ray diffractometer with Cu Kα radiation (1.5418 Å).

14. A process for preparing the compound of formula (VII-HBr) of claim 10, 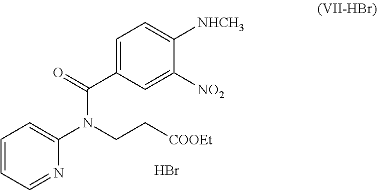 [SMILES: CCOC(=O)CCN(C(=O)c1ccc(NC)c([N+](=O)[O-])c1)c1ccccn1 ; Br]
the process comprising reacting the compound of formula (VII) with hydrobromic acid and isolating the product obtained as a solid.

15. The process according to claim 14, wherein the compound of formula (VII) is obtained by reacting the compound of formula (IX), 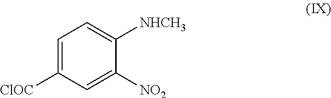
with the compound of formula (VIII), 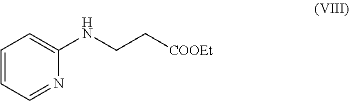 [SMILES: CCOC(=O)CCNc1ccccn1]
in the presence of a base.

16. The process according to claim 3, wherein the compound of formula (VII-HBr) is isolated as a solid.

17. The process according to claim 16, wherein the amount of inorganic base in step a) is 2-8% by weight in relation to the starting compound of formula (VII).

18. The process according to claim 16, wherein the inorganic base of step a) is K₂CO₃ or K₃PO₄.

19. The process according to claim 16, wherein the conversion of compound of formula (VI) into a compound of formula (I) or a pharmaceutically acceptable salt thereof is carried out by the following steps:
(b1) the compound of formula (VI) is reacted with the compound of formula (V) 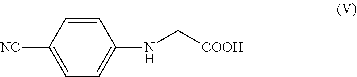 [SMILES: N#Cc1ccc(NCC(=O)O)cc1]
in the presence of a coupling agent and subsequent cyclization with a cyclization agent to yield the compound of formula (IV) 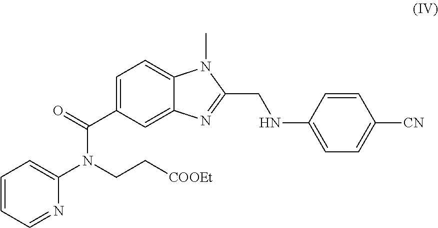 [SMILES: CCOC(=O)CCN(C(=O)c1ccc2c(c1)nc(CNc1ccc(C#N)cc1)n2C)c1ccccn1]
and, optionally, the compound of formula (IV) is converted into a salt thereof by reaction with the corresponding acid;
(b2) the product obtained in step (b1) is reacted in the presence of hydrochloric acid and ethanol, and subsequently ammonia or an ammonium salt is added to yield the compound of formula (II), 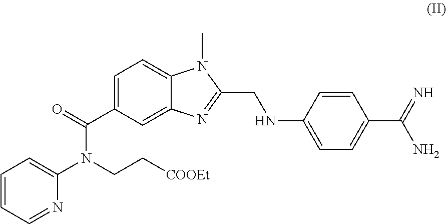 [SMILES: CCOC(=O)CCN(C(=O)c1ccc2c(c1)nc(CNc1ccc(C(=N)N)cc1)n2C)c1ccccn1]
and, optionally, the compound of formula (II) is converted into a salt thereof by reaction with the corresponding acid;
(b3) the compound of formula (II) or a salt thereof is converted into a compound of formula (I) by a hydrolysis reaction or by reaction with a n-hexyl haloformate in the presence of a base; and
(b4) optionally the compound of formula (I) is converted into a pharmaceutically acceptable salt thereof by treatment with an acid, or either a pharmaceutically acceptable salt of the compound of formula (I) is converted into a compound of formula (I) by treatment with a base, or either a salt of the compound of formula (I) is converted into another salt of the compound of formula (I) by ion exchange.

20. The process according to claim 1, wherein the inorganic base of step a) is K₂CO₃ or K₃PO₄.
